(19) United States
(12) Patent Application Publication
(10) Pub. No.: US 2025/0322920 A1
(43) Pub. Date: Oct. 16, 2025

(54) COMPUTER-IMPLEMENTED METHOD FOR DETERMINING THE STATES IN VIVO AND IN VITRO BY ANALYZING THE BLOOD PARAMETERS MEASURED IN A HEMATOLOGICAL ANALYSIS DEVICE

(71) Applicant: RobotDreams GmbH, Graz (AT)

(72) Inventors: Dimitrij SHULKIN, Graz (AT); Aleksandr Pushkin, Graz (AT); Ulrich Weigelt, Graz (AT)

(21) Appl. No.: 19/252,752

(22) Filed: Jun. 27, 2025

Related U.S. Application Data
(63) Continuation of application No. PCT/AT2024/060119, filed on Apr. 4, 2024.

(30) Foreign Application Priority Data
Apr. 5, 2023 (DE) 10 2023 108 748.7

(51) Int. Cl. G16H 10/40; G01N 33/49; G06N 20/20; G16H 15/00

(52) U.S. Cl. CPC G16H 10/40; G01N 33/492; G06N 20/20; G16H 15/00

(57) ABSTRACT

A computer-implemented method for determining states in vivo and in vitro by analyzing blood parameters, including obtaining blood parameters of a blood sample by a hematology analyzer, the blood parameters including quantitative and qualitative measurement variables, and the measurement variables include properties of individual cells, and the individual cells comprise blood cells. The computer-implemented method further includes creating a scatterplot having at least two axes, and each axis of the scatterplot comprises a different measurement variable; and determining an in-vivo and/or in-vitro and/or post-mortem state by a deep learning model and/or a machine learning model. The input variable for the deep learning model includes a scatterplot, and the input variable for the machine learning model includes 1D vector. The 1D vector is created by vectorizing the scatterplot; and automatically generating a report including the result regarding the determination of the state.

The present application is a continuation of International Application PCT/AT2024/060119 filed on Apr. 4, 2024. Thus, all of the subject matter of International Application PCT/AT2024/060119 is incorporated herein by reference.

### TECHNICAL FIELD

The present invention relates to the field of medical diagnostics, in particular to the analysis of blood samples and the determination of states in vivo and in vitro. The invention uses advanced techniques from artificial intelligence (AI), such as deep learning and machine learning, for the automated analysis of blood parameters measured in a hematology analyzer in order to carry out precise and efficient diagnoses and state determinations.

### BACKGROUND OF THE INVENTION

Hematology analyzers are widely used diagnostic tools used to test blood samples in clinical laboratories. These analyzers measure various parameters of blood cells, such as the number, size, volume and complexity of the cells. Analysis of these blood parameters allows health care practitioners to obtain information about a patient's health status, possible disorders, or other relevant characteristics.

However, traditional approaches to analyzing hematology parameters are often time-consuming and require manual interpretation by skilled personnel.

### PRIOR ART

Each complete blood count measures high-dimensional single-cell information, but clinical decisions are currently based on few derived statistics. The enormous potential of the full set of blood cell measurements was well estimated, and previous efforts attempted to achieve early detection of infections by identifying immature granulocytes or a prognosis for some malignancies by counting the number of WBCs with atypical features (Statland B E, Winkel P, Harris S C, Burdsall M J, Saunders A M. Evaluation of biological sources of variation of leukocyte counts and other hematologic quantities using very precise automated analyzers. Am J Clin Pathol. 1978 January; 69 (1): 48-54. doi: 10.1093/ajcp/69.1.48. PMID: 563672.) These efforts have had limited impact but indicate the potential for improved clinical decision support (Gijsberts C M, Ruijter H M, de Kleijn D P V, Huisman A, Ten Berg M J, van Wijk R H A, Asselbergs F W, Voskuil M, Pasterkamp G, van Solinge W W, Höfer I E. Hematological Parameters Improve Prediction of Mortality and Secondary Adverse Events in Coronary Angiography Patients: A Longitudinal Cohort Study. Medicine (Baltimore). 2015 November; 94 (45): e1992. doi: 10.1097/MD.0000000000001992. PMID: 26559287; PMCID: PMC4912281).

In this study [Campuzano-Zuluaga G, Alvarez-Sánchez G, Escobar-Gallo G E, Valencia-Zuluaga L M, Ríos-Orrego A M, Pabón-Vidal A, Miranda-Arboleda A F, Blair-Trujillo S, Campuzano-Maya G. Design of malaria diagnostic criteria for the Sysmex XE-2100 hematology analyzer. Am J Trop Med Hyg. 2010 March; 82 (3): 402-11. doi: 10.4269/ajtmh.2010.09-0464. PMID: 20207864; PMCID: PMC2829900.], the authors used scatterplots generated by the Sysmex XE-2100 hematology analyzer to distinguish blood samples from patients with malaria from those without. In the study, the authors analyzed the scatterplots and identified specific patterns that occurred in malaria-infected patients. Based on these patterns, they developed diagnostic criteria to detect malaria infections. By analyzing the scatterplots and applying the developed criteria, they were able to achieve a high sensitivity and specificity in the malaria diagnosis.

The study [Chaudhury A, Noiret L, Higgins J M. White blood cell population dynamics for risk stratification of acute coronary syndrome. Proc Natl Acad Sci USA. 2017 Nov. 14; 114 (46): 12344-12349. doi: 10.1073/pnas.1709228114. Epub 2017 Oct. 27. PMID: 29087321; PMCID: PMC5699055.] investigates the dynamics of white blood cells in relation to acute coronary syndrome (ACS) and identifies specific clusters of lymphocytes, neutrophils and monocytes using an Abbott-Cell-Dyn-Sapphire hematology analyzer. Using scatterplots, these clusters are analyzed using the Fokker-Plank differential equation to achieve risk stratification of healthy patients and patients with ACS. The mathematical model achieves an accuracy of over 70% in identifying patients who initially had negative screening tests but were diagnosed with ACS within 48 hours.

In contrast, the Pushkin and Shulkin RU 2733077 C1 patent describes a method for diagnosing ACS based on the measured properties of white blood cells. Scatterplots of cell size and cell complexity are produced, which are also measured by a hematology analyzer of the Abbott-Cell-Dyn-Sapphire brand. Clusters of lymphocytes, monocytes, and neutrophils are manually identified and grouped into 1D vectors. These are then reduced by a principal component analysis and used for analysis by multi-layer perceptrons (MLPs). The method is evaluated using a small database of 211 measurements, achieving a sensitivity of 0.97 and a specificity of 0.94 (AUC=0.96).

The study [Pushkin A S, Shulkin D, Borisova L V, Akhmedov T A, Rukavishnikova S A. [Algorithm to stratify the risk of myocardial infarction in patients with acute coronary syndrome at primary examination.] Klin Lab Diagn. 2020; 65 (6): 111-222. Russian doi: 10.18821/0869-2084-2020-65-6-111-222. PMID: 32459900)] investigates the use of the method described in the above-mentioned patent RU 2733077 C1 for the classification of myocardial infarction and unstable angina pectoris in patients with acute coronary syndrome (ACS). The authors created a small database of 307 anonymized measurements taken with an Abbott-Cell-Dyn-Sapphire hematology analyzer. Of these, 214 measured data were used for training and 93 for evaluation of the method. The results showed a sensitivity of 0.77 and a specificity of 0.80 (AUC=0.77) for the classification of myocardial infarction and unstable angina pectoris in patients with ACS.

The characterization of acute leukemias by various hematological analyzers has been documented previously. Krause J R et al. evaluated the use of Technicon H-1 (Technicon Instruments Corporation, Tarrytown, NY, USA) to characterize acute leukemias. They were able to distinguish between acute myeloid leukemia (AML) and acute lymphoblastic leukemia (ALL) based on myeloperoxidase activity and the core characteristics of the cells. In AML, they pointed out that the AML of the Franco-American-British (FAB) type—M3, M4 and M5—have characteristic cytograms. Chronic myeloid leukemia (CML) also exhibited a characteristic pattern [Krause J R, Costello R T, Krause J, Penchansky L. Use of the Technicon H-1 in the characterization of leukemias. Arch Pathol Lab Med 1988; 112:889-4]. Similarly, Kawarabayashi et al. investigated the utility of Technicon H-1 (Technicon Instruments Corporation, Tarrytown, NY, USA) for the detection of blast cells. [Kawarabayashi K, Tsuda I, Tatsumi N, Okuda K. Leukemic blasts detected by the Technicon H-1® blood cell counter. Am J Clin Pathol 1987; 88:624-27.].

Hoyer et al. examined the ability of the hematology analyzer Coulter STKS to distinguish acute leukemias. They concluded that the use of a number of suspicious or definitive flags as screening criteria for microscopic examination would be the best approach to correctly identify leukemias. They also concluded that scatterplot patterns are not meaningful for the classification of acute leukemias. [Hoyer J D, Fisher C P, Soppa V M, Lantis K L, Hanson C A. Detection and classification of acute leukemia by the Coulter STKS Hematology Analyzer. Am J Clin Pathol 1996; 106:352-8.] Bruno et al. 1994 and Pettit et al. 1995 also examined the scatterplot pattern of acute leukemias with the Coulter STKS hematology analyzer [Bruno A, Del Poeta G, Venditti A, Stasi R, Adorno G, Aronica G, et al. Diagnosis of acute myeloid leukemia and system Coulter VCS. Haematologica 1994; 79:420-8.], [Pettitt A R, Grace P, Chu P. An assessment of the Coulter VCS automated differential counter scatterplots in the recognition of specific acute leukaemia variants. Clin Lab Haematol 1995; 17:125-9]. Virk et al. investigated the usefulness of the cell population data (VCS parameters) of the automated hematology analyzer Coulter LH 780 as a rapid screening tool for AML in resource-constrained laboratories. They concluded that the cell population data together with the scatterplots can provide a cost-effective and rapid initial diagnosis of acute leukemias. These parameters can then be used to distinguish malignant hematological diseases from non-malignant ones [Virk H, Varma N, Naseem S, Bihana I, Sukhachev D. Utility of cell population data (VCS parameters) as a rapid screening tool for Acute Myeloid Leukemia (AML) in resource-constrained laboratories. J Clin Lab Anal 2019; 33: e22679].

In the study [Aparna N et al, Scattergram patterns of hematological malignancies on Sysmex XN-series analyzer, Journal of Applied Hematology, 2021, 12, 2, 83-39], the scatterplots of various primary hematological malignancies associated with the use of the Sysmex XN hematology analyzer are investigated. The authors have conducted a retrospective study in which they have collected the details of 291 newly diagnosed cases of hematological malignancies and 48 cases of leukemoid reactions. The aim of the study was to find out whether specific hematological malignancies produce specific scatterplot patterns. The authors have found that different patterns of scatterplots have been observed, and these patterns can be used to distinguish between different reactive and neoplastic states. The pattern analysis confirms that all cases examined have individual patterns. These patterns can be used for targeted investigation of cases for further molecular and cytogenetic analysis.

The studies and patents mentioned above focus mainly on manual, statistical and mathematical methods and machine learning, such as principal component analysis and multi-layer perceptrons (MLPs). Deep learning models, especially CNNs, have achieved excellent results in many areas in recent years, including medical imaging and diagnosis [Litjens G, Kooi T, Bejnordi B E, Setio A A A, Ciompi F, Ghafoorian M, van der Laak J A W M, van Ginneken B, Sánchez C I. A survey on deep learning in medical image analysis. Med Image Anal 2017 December; 42:60-88. doi: 10.1016/j.media.2017.07.005. Epub 2017 Jul. 26. PMID: 28778026.].

US 2018/0247715 A1 describes a method for the diagnosis and characterization of cancer by means of artificial neural networks (ANN) by analyzing white blood cells by means of a flow cytometer.

The publications discussed above focus primarily on methods for diagnosing diseases in humans.

### OBJECT OF THE INVENTION

The object of the invention is to provide a method for determining states and/or automatically producing a report on states based on the analysis of blood parameters, which is distinguished from the prior art by an increased accuracy, a broader applicability and a lower expenditure of labor.

### DESCRIPTION OF THE INVENTION

The object is achieved by a computer-implemented method for determining states in vitro and in vivo by analyzing blood parameters measured in a hematology analyzer, wherein the method comprises:
a) obtaining blood parameters of a blood sample by means of a hematology analyzer, wherein the blood parameters comprise quantitative and qualitative measurement variables, wherein the measurement variables comprise properties of individual cells, wherein the individual cells comprise blood cells, wherein the blood cells comprise white blood cells, red blood cells and platelets, wherein the white blood cells comprise monocytes, lymphocytes, basophils, eosinophils and neutrophils;
b) creating at least one scatterplot having at least two axes, wherein each axis of the scatterplot comprises a different measurement variable;
c) determining at least one in vivo and/or in vitro and/or post mortem state by means of at least one deep learning model and/or a machine learning model, wherein the input variable for the at least one deep learning model comprises at least one scatterplot, wherein the input variable for the at least one machine learning model comprises at least one 1D vector, wherein the 1D vector is created by vectorizing the at least one scatterplot; and
d) automatically generating a report which comprises at least one result regarding the determination of the at least one state.

The object is also achieved by a computer-implemented method for automatically creating a report regarding the determination of states in vitro and in vivo by analyzing blood parameters measured in a hematology analyzer, wherein the method comprises:
a) obtaining blood parameters of a blood sample by means of a hematology analyzer, wherein the blood parameters comprise quantitative and qualitative measurement variables, wherein the measurement variables comprise the properties of individual cells, wherein the individual cells comprise blood cells, wherein the blood cells comprise white blood cells, red blood cells and platelets, wherein the white blood cells comprise monocytes, lymphocytes, basophils, eosinophils and neutrophils;
b) creating at least one scatterplot with at least two axes, each axis of the scatterplot comprising a different measurement variable;
c) determining at least one in vivo and/or in vitro and/or post mortem state by means of at least one deep learning model and/or machine learning model, wherein the input variable for the at least one deep learning model comprises at least one scatterplot, wherein the input variable for the at least one machine learning model comprises at least one 1D vector, wherein the 1D vector is created by vectorizing the at least one scatterplot;
d) automatically generating a report which comprises at least one result regarding the determination of the at least one state; and
(e) transmitting the report by means of a data carrier signal
(f) receiving the transmitted report

The present invention relates to a computer-implemented method for determining states in vivo and in vitro by analyzing blood parameters measured in a hematology analyzer and for automatically generating a report on the determined states. The method uses AI techniques, in particular deep learning and machine learning models, for automated analysis and interpretation of the measured blood parameters and for determining the states.

The approaches described in the prior art require numerous manual processing steps, such as cluster analysis, in order to identify the subpopulations of white blood cells in scatterplots. In the invention, the cluster analysis is not necessary to determine the states, because all components of the blood of the blood sample are taken into account in any case, in particular also red blood cells and platelets. Deep learning models are capable of automatically recognizing the critical patterns and structures for state determination during the training phase. In addition, traditional cluster analysis methods are often not feasible with multi-dimensional scatterplots. By using deep learning models, manual processing steps can be reduced or even eliminated, enabling more efficient and accurate state determination. These models are able to automatically capture and learn complex patterns and relationships within the data, speeding up the analysis process and improving the accuracy of results.

The invention enables improved analysis and interpretation of hematology parameters, which leads to increased accuracy and efficiency in the determination of states in vivo and in vitro. In addition, the invention can contribute to reducing the workload of healthcare professionals, since automated analysis and reporting minimizes the manual effort of interpretation. The use of AI techniques also allows continuous improvement of models by adding new data and experiences, optimizing diagnostic performance over time. The method according to the invention relates to the examination of blood samples from humans and animals.

The invention can use various deep learning and machine learning models, including Convolutional Neural Networks, Recurrent Neural Networks, Support Vector Machines, Decision Trees, and Random Forests, to analyze different aspects of the measured blood parameters and combine the results. These models can be combined in an ensemble approach to improve predictive accuracy and robustness of AI by combining different models or model instances to make a consolidated prediction of the state.

A further advantage of the invention is that it is able to determine a plurality of states which can occur in vivo, ex vivo, in vitro and/or post-mortem. This comprises states and processes both inside and outside a living organism, including changes in blood cell morphology, cell composition, cell function or other characteristics of the individual cells as a result of storage, handling and/or analysis.

The invention can be used for various fields of application, such as, for example, in clinical diagnostics, research, forensic analysis and veterinary examination. By integrating the invention into existing hematology analyzers or laboratory systems, the diagnostic capabilities of these systems can be expanded and the quality of patient care improved.

The invention can help reduce the time required to analyze blood samples and produce reports, thereby increasing the efficiency of laboratories and reducing the cost of patient care.

Overall, the present invention offers an innovative solution for improving hematology analysis and state determination in vivo and in vitro by the use of artificial intelligence. The invention enables an automated, precise and efficient analysis of blood parameters from hematology analyzers and can contribute to improving the quality of patient care and increasing the efficiency of laboratories.

A blood sample is taken from the subject to be examined, for example the first venous whole blood is taken from a cubic vein, for example with the aid of a 4 ml vacuum system for blood withdrawal into an e.g. Vacutest tube (KIMA, Italy) and applied to the inner surface of the e.g. 7.2 mg K3EDTA tube walls. Other variants are conceivable. This sample is then used to determine diseases or other states by the method according to the invention. Sampling is not part of the method.

The tube may be stirred after blood collection by turning it upside down and turning it horizontally and vertically for 30 seconds. Thereafter, the clinical blood test is performed in an open mode on an automatic hematology analyzer, e.g. CELL-DYN Sapphire (Abbott Laboratories, USA). In this case, the individual cells of the whole blood count are measured in a high-dimensional manner, the measurements comprising the properties of the individual cells, as shown by way of example in FIG. 1.

The measurements are copied from the analyzer, e.g. as FCS files or in another format, and transferred to an accessible PC or mobile computer device or a cloud for machine processing. These measurements include blood parameters as properties of leukocytes, wherein leukocytes include neutrophils, eosinophils, basophils, lymphocytes, and monocytes, wherein the properties include size, granularity, lobularity, and complexity.

### A) Complete Blood Count

Blood cells in the bloodstream of a human or an animal continuously pass through almost all tissues in vivo at high speed, and their common degree of maturity, state of activation, proliferation and senescence reflect the current pathophysiological or health state: healthy rest, acute reaction to pathology, chronic compensation for disease and ultimately decompensation. The complete blood counts include measurement of single cell characteristics for tens of thousands of blood cells and provides an overview of these states. Complete blood count includes measurement of white blood cells, red blood cells, and platelets. Complete blood count is a common blood test that is often part of a routine examination. A complete blood count can help identify a variety of disorders, such as infections, anemia, immune system disorders, and blood cancers. [MedlinePlus Medical Encyclopedia. (2021) Complete blood count (CBC). U.S. National Library of Medicine. Retrieved from https://medlineplus.gov/Lab-tests/complete-blood-count-cbc/].

### B) Measurement of Blood Parameters in the Hematology Analyzer

Complete blood count is usually done with an automated laboratory device called a hematology analyzer. It uses different technologies to measure the different blood cells and blood parameters contained in a complete blood count. One of the most common technologies used in hematology analyzers is impedance measurement. In this procedure, the blood sample is passed into a tiny chamber filled with a conducting fluid. Electrical pulses are then passed through the liquid, measuring the resistance produced by the various blood cells. Based on these measurements, the device can determine the number and size of red blood cells, white blood cells, and platelets.

Another method used in hematology analyzers is laser scattered light analysis. The blood sample is passed through a thin beam of laser light that is reflected or scattered by the various blood cells. The reflected light is then captured by photodetectors, which can detect the size, shape and complexity of the various cells.

Most modern hematology analyzers combine these two technologies to achieve higher accuracy and reliability. The blood sample is directed into several channels, each of which is intended for a specific analysis. The device can then automatically detect and quantify the different cell types and display the results in a report. Only the raw measurement data is generated in the measurement channels of the hematology analyzer. These data contain information such as the size, shape, density or color of the blood cells, which are captured by the specific measurement methods in each measurement channel. The raw measurement data from the measurement channels are then processed by the analyzer's software and usually converted into a set of results and blood parameters. These results and blood parameters include the total number of white and red blood cells, hematocrit, hemoglobin concentration, and other relevant information about blood cells. The hematology analyzer software usually performs complex algorithms and statistical methods to obtain more accurate results from raw measurement data.

### C) Analysis of Scatterplots

One of the most important tools for representing blood cell characteristics measured by a hematology analyzer is a scatterplot.

Scatterplots obtained from hematology analyzers can also be used for states other than diseases such as post-harvest blood age, as well as for other living organisms such as animals.

A scatterplot is a graphical representation of data points that are arranged on a two-dimensional plane. Each point in the diagram represents a single cell, and the two axes represent different blood parameters measured by the analyzer. In general, most hematology analyzers will produce two basic scatterplots, one for red blood cells (RBC) and one for white blood cells (WBC). The RBC scatterplot shows the size and distribution of red blood cells, while the WBC scatterplot shows the size and distribution of different types of white blood cells. Other scatterplots may be available depending on the hematology analyzer model and blood parameters required. Using these scatterplots, doctors and health care practitioners can manually identify or suspect various states that have characteristic patterns. Most modern hematology analyzers have the ability to store the collected measurement data in a digital form. This data can then be exported and used for further analysis and visualization, including the creation of scatterplots.

### D) Deep Learning Analysis to Determine States In Vitro and In Vivo

The present description describes a computer-implemented method for determining states in vivo and in vitro by analyzing blood parameters measured with a hematology analyzer using artificial intelligence, with deep learning models being used. In an exemplary application, it is demonstrated for the first time that the proposed method is capable of successfully classifying the blood age in vitro after sampling.

### DESCRIPTION OF EMBODIMENTS

Exemplary embodiments of the method according to the invention are explained below.

By way of example, the blood age in a blood sample after blood collection is to be determined as an in vitro state. The age of the blood in vitro after blood collection may be of interest, as some studies have shown that it has an effect on the quality and effectiveness of the red blood cells transfused. Some properties of red blood cells are thought to change over time, including viscosity, ability to transport oxygen, and expression of antigens on the surface of cells. One possible application of knowledge about the age of the blood could be to reduce transfusion-related morbidity and mortality by selecting the red blood cells of the most appropriate age. For example, the use of fresh blood in certain patients, such as trauma patients or patients with severe bleeding, may be beneficial to maximize the effectiveness of the transfusion and reduce complications. However, determining the age of blood after blood sampling is not easy, and there is no standardized method to do so. There are various approaches and techniques to estimate the age of the blood, including measuring the expression of certain proteins on the surface of red blood cells and analyzing changes in the red blood cell membrane over time.

The blood parameters of a blood sample are obtained in a modern hematology analyzer. There are several manufacturers of hematology analyzers on the market. Some of the most well-known and commonly used brands include:
Sysmex: Sysmex is one of the largest providers of hematology analyzers worldwide. The company offers a wide range of devices suitable for various applications, from small clinics to large laboratories.
Beckman Coulter: Beckman Coulter is another leading provider of hematology analyzers, offering a wide range of devices suitable for various applications, from routine analysis to specialized research purposes.
Abbott Laboratories: Abbott Laboratories is a large company that manufactures a variety of medical devices and diagnostics, including hematology analyzers.
Siemens Healthcare Diagnostics: Siemens is a well-known manufacturer of medical devices and also offers hematology analyzers.
Roche Diagnostics: Roche is a global diagnostics company and also offers a range of hematology analyzers.
Horiba Medical: Horiba is a global medical device manufacturer that also offers a range of hematology analyzers.
Mindray: Mindray is a Chinese company that offers a wide range of medical devices and solutions, including hematology analyzers.
Nihon Kohden: Nihon Kohden is a Japanese manufacturer of medical devices, including hematology analyzers.
Boule Medical: Boule Medical is a Swedish company specializing in the development of hematology analyzers.
Diatron: Diatron is a Hungarian manufacturer of medical devices, including hematology analyzers.
HemoCue: HemoCue is a Swedish company specializing in the development of point-of-care tests and analyzers, including hematology analyzers.
Shenzen Mindray Bio-Medical Electronics Co.: Shenzen Mindray is a Chinese company that offers a wide range of medical devices and solutions, including hematology analyzers.
Human Diagnostics: Human Diagnostics is a German company specializing in the development and manufacture of in vitro diagnostics and laboratory equipment, including hematology analyzers.
Erba Mannheim: Erba Mannheim is a global company specializing in the development of diagnostics and medical devices for various applications, including hematology analyzers.
Heska: Heska is an American company specializing in the development of diagnostic and treatment solutions for pets, including hematology analyzers

In a blood sample which is examined by means of a hematology analyzer, various types of blood cells can be detected and taken into account by the method according to the invention. Blood cells and their functions comprise:
Erythrocytes (Red blood cells): These cells carry oxygen from the lungs to the tissues and carbon dioxide from the tissues to the lungs. The state of red blood cells can be influenced by various factors, such as the oxygen content of the blood, the pH value and the amount of carbon dioxide.
Leucocytes (White blood cells): These cells play an important role in fighting infections and fighting foreign substances in the body. The state of white blood cells may be affected by the degree of inflammation, the type of pathogen, and other factors.
Lymphocytes: This type of white blood cell plays an important role in the immune system and can occur in various states, depending on the circumstances. For example, they can be activated to fight infections or they can remain inactive when there is no threat to the body.
Monocytes: These cells are also white blood cells and play a role in fighting infections and foreign substances. They can transform into different types of tissue macrophages that phagocytize foreign substances and protect the body from damage.
Basophils: This type of white blood cell is involved in allergic reactions and can occur in various states, depending on the type of allergic reaction and other factors.
Neutrophils: These cells are the most common type of white blood cell and play an important role in fighting infection. They may be in various states, depending on the type of infection and other factors.
Eosinophils: This type of white blood cell is involved in the control of parasites and allergies and can occur in various states, depending on the circumstances.
Thrombocytes (Platelets): These cells play an important role in blood clotting. The state of platelets can be affected by various factors such as inflammation, infections and other disorders of the blood system.

In addition, other cell types and/or particles may be present in the blood sample, the properties of which can likewise be detected by hematology analyzers. These cell types and/or particles can also be taken into account by the method according to the invention. These cells and/or particles may be of clinical interest and comprise, but are not limited to:
Blasts: Immature blood cells normally present in the bone marrow and entering the blood in small amounts. An increased number of blasts in the blood may indicate a blood disorder such as leukemia.
Cell fragments: fragments of cells resulting from cell damage or degradation. For example, schistocytes (broken red blood cells) may occur in certain disorders, such as hemolytic-uremic syndrome or thrombotic-thrombocytopenia purpura.
Circulating tumor cells (CTCs): Cancer cells that separate from the primary tumor and enter the bloodstream. CTCs can occur in various cancers and are a potential marker for tumor metastasis.
Endothelial cells: cells that form the inner lining of blood vessels. An increased number of endothelial cells in the blood may indicate inflammation or injury of blood vessels.
Microparticles: Small cell fragments secreted by different cell types that circulate in the blood. They may occur in inflammation, coagulation disorders, or other disorders.

In some cases, hematology analyzers can also detect parasites in the blood. Some blood parasites, such as those that cause malaria (Plasmodium spp.), can be found within red blood cells (erythrocytes). If the infection is severe, these infected cells can be detected by the analyzers and possibly identified as abnormal cells.

The characteristics or measured variables may vary according to the type and technology of analyzer used and may include:
Number of cells
Size: The size of cells can help distinguish different cell types because they usually have different sizes. For example, red blood cells are smaller than white blood cells.
Shape: The shape of the cells can also contribute to identification. Red blood cells are usually disk-shaped, whereas white blood cells and platelets may have different shapes.
Volume: The volume of cells can be related to the diameter of the cell and varies for different cell types.
Granularity or complexity: The internal structure of cells, also called granularity, can be used to identify cell types, particularly leukocytes, which can be divided into different subgroups based on their granularity (eg, granulocytes and lymphocytes).
Electrical conductivity: The electrical conductivity of the cells can also be used to differentiate between cell types because they have different conductivities due to their different membrane structures and cellular contents.
Light scattering: Some hematology analyzers use light scattering caused by the cells to evaluate size, shape, and granularity. Forward scattering is proportional to the size of the cell, while side scattering is related to the granularity of the cell.
Mean Corpuscular Volume (MCV)
Mean corpuscular hemoglobin (MCH)
Mean corpuscular hemoglobin concentration (MCHC)
Red blood cell distribution width (RDW)
Mean platelet volume (MPV)
Platelet distribution width

Measurements of the properties of the cells are transferred from the hematology analyzer to an accessible PC, mobile computer, mobile device or cloud for machine processing and subsequent AI-based analysis. The measurement results from hematology analyzers can be transmitted in different formats, depending on which interfaces the analyzer supports and which formats the target information system accepts. Formats for the transfer of measurement results from hematology analyzers shall include:
HL7 (Health Level Seven): This is a standard format for the exchange of clinical and administrative data between different medical information systems. Hematology analyzers can transmit measurement results in HL7 format to an information system via an interface such as a TCP/IP connection.
ASTM (American Society for Testing and Materials): ASTM is another format for the exchange of data between medical devices and information systems. Hematology analyzers can transmit measurement results in ASTM format to an information system via an RS-232 interface. Text files: The measurement results can be saved and transferred as text files, which can be in various formats such as CSV, TXT or XML. These files can then be processed in other programs such as spreadsheets or databases.
DICOM: DICOM is a standard format for transferring medical images and other data between different systems and devices. Some hematology analyzers can create DICOM files that can then be used in image processing programs or electronic health records.
FCS: FCS stands for “Flow Cytometry Standard” and is a file format used in flow cytometry to store data from individual cells or particles. The FCS format is an open standard developed by the International Society for Advancement of Cytometry (ISAC) to ensure compatibility between different flow cytometry devices and software platforms.

Machine processing consists of the automatic creation of at least one scatterplot. Each point in the diagram represents a single cell, and the two axes represent different properties measured by the analyzer. In a scatterplot based on data from a hematology analyzer, two properties of the measured cells are often represented along the x- and y-axes. Typical properties represented in scatterplots comprise:
Size (cell diameter or volume): The size of cells can be represented on the x or y axis to distinguish different cell types from each other because they have different sizes.
Granularity or internal complexity: The granularity of cells can be represented on the x- or y-axis and is commonly used to identify subsets of leukocytes, such as lymphocytes, monocytes, and granulocytes.
Light scattering: Forward scattering and side scattering can be represented in scatterplots. Forward scattering correlates with the size of cells, while side scattering provides information about the granularity or internal complexity of cells.

In addition to at least one scatterplot in simple form, the following can also be created and used as the at least one scatterplot:
A 3D scatterplot: In a 3D scatterplot, three axes (x, y, and z) are used to represent three properties simultaneously.
A scatterplot matrix: This method displays multiple 2D scatterplots in a matrix, each diagram representing a pair of properties.
Parallel Coordinates: In parallel coordinates, several vertical axes are arranged parallel to each other, each axis representing a dimension or property. The data points are represented by lines connecting the corresponding values on each axis.
A histogram: A histogram shows the distribution of the measured values of a particular property or feature. For example, when analyzing blood cells, one could create histograms for size, granularity, or fluorescence intensity.
A density diagram: this corresponds to a scatterplot using an additional dimension, such as different colors for the points of the scatterplot; in this way, points lying one on top of the other can also be distinguished from one another and taken into account.

Subsequently, at least one scatterplot is used as an input variable for at least one deep learning model which is based on artificial neural networks. This means that it consists of many layers of neurons and can learn a hierarchical representation of data and is able to extract complex features from large amounts of data and make precise predictions of the state based on these features. The at least one deep learning model may include at least one deep learning model from the following group:
Convolutional Neural Networks (CNNs): CNNs are a powerful deep learning model designed specifically for image processing. They can analyze both 2D and 3D images and can be used for a variety of applications, such as image recognition, object recognition, facial recognition and medical imaging.
Generative Adversarial Networks (GANs): GANs are a pair of artificial neural networks that work together to generate new images that look similar to the images in the training dataset. A network called a generator generates new images, while another network called a discriminator attempts to distinguish between real and generated images. GANs are often used for the creation of artificial images, image restoration and reconstruction.
Recurrent Neural Networks (RNNs): RNNs are another deep learning model that can be used to process multi-dimensional images.
Long Short-term memory networks
Transformer Networks
3D Convolutional Neural Networks (3D CNNs): 3D CNNs are an extension of CNNs that can be used to process 3D images.
4D Convolutional Neural Networks

The scatterplot can optionally be processed before the deep learning analysis (data pre-processing), wherein the processing includes:
Resizing: Resize images to the appropriate size for the model, such as 224×224 for many CNN models.
Normalization: The pixel values of the images should be normalized by scaling them to a range between 0 and 1 to ensure that they are comparable for the model.
Standardization
Noise reduction: It may be necessary to remove noise from the images, especially if the images are of poor quality or are highly compressed.
Test Time Augmentation (TTA)
Clustering
Contrast adjustment
Filtering
Cropping: It may be necessary to remove unwanted areas of the image to ensure that the model only gets relevant features.

These processing operations can be applied individually or in combination.

In parallel, the scatterplot can be converted into a one-dimensional vector (1D vector) in order to use this as an input variable for at least one machine learning model which can only process one-dimensional data structures. The method comprises a vectorization step in which the data of the scatterplot are converted into a one-dimensional vector. The vectorization takes place by combining the data in a specific order, so that the resulting vector represents a unique representation of the scatterplot. Vectorization could comprise:
Flattening: This technique refers to the simple conversion of a multidimensional matrix into a one-dimensional vector by the concatenation of the individual rows or columns of the matrix. In image processing, this method can be used, for example, by arranging the pixel values of a two-dimensional matrix in a single-dimensional vector in simple succession.
Reshaping: Reshaping refers to changing the shape of a multidimensional matrix without changing the individual elements of the matrix. In image processing, reshaping can be used, for example, to convert a multidimensional image into another format, such as a larger or smaller two-dimensional matrix or tensor. Reshaping can also be used in other areas of data analysis and machine learning to bring multidimensional data into a different structure that is better suited to certain algorithms.
Zigzag scanning: This technique is commonly used in JPEG compression of images. In this case, the pixel values of a matrix are scanned in a “zigzag” pattern, so that the pixel values of the resulting one-dimensional vector are not arranged in the order of the original matrix.
PCA-based techniques: Another method of vectorizing multidimensional data is to use Principal Component Analysis (PCA) or other linear transformations. This transforms the original matrix into a new base that contains the most important features of the original data set. The data can then be represented as linear combinations of the new base vectors, which allows a more efficient and compact representation.

Suitable machine learning models that can process one-dimensional data structures include, for example:
K-Nearest Neighbors (KNN): KNN models are suitable for processing one-dimensional data structures in which each point is represented by a series of numerical or categorical features. The model finds the K closest neighbors to a given point and uses them to make predictions about the class or value of the point.
Support Vector Machines (SVM): SVM models are suitable for processing one-dimensional data structures where each feature is numerical. SVMs are especially useful when the data is two classes because they seek a linear separation between the classes and make a prediction based on that separation.
Decision Trees: Decision trees are also useful for processing one-dimensional data structures where each feature is numeric or categorical. The model recursively divides the feature space into partitions by making decisions about the values of the features.
Multi-Layer Perceptron (MLP): MLPs are neural network models designed primarily for processing one-dimensional data structures. Each layer of the MLP consists of a series of neurons, each equipped with a series of weights and an activation function to make predictions. MLPs can be used for classification and regression problems.
Adaboost: Adaboost is a machine learning algorithm that is suitable not only for one-dimensional data structures, but also for multi-dimensional data structures. Adaboost is an ensemble model consisting of a series of weak learners trained one after the other. Each weak learner is trained on a portion of the data and then the data is weighted to focus on the previous learner's mistakes. By combining multiple weak learners, Adaboost can achieve high accuracy in predicting classes or values in complex multidimensional data structures.
Gradient boosting: The gradient boosting method is an ensemble model that uses a set of weak learning algorithms to create a powerful model. By combining several weak learning algorithms, the gradient boosting method can usually achieve a higher accuracy than other machine learning models such as decision trees, naive Bayes or support vector machines.
Naive Bayes: Naive Bayes is a probabilistic model that is suitable for the processing of one-dimensional data structures in which each feature is independent of the others. It works by calculating the probability that an instance belongs to a particular class, based on the distribution of features in that class.
One-Class Support Vector Machines (OCSVM): One-Class Support Vector Machines (OCSVM) are a special form of Support Vector Machines (SVM) that can be used for anomaly detection in data. Unlike traditional SVMs, which are used to classify data into two or more classes, OCSVMs are used to discover data in a single class. The goal of an OCSVM model is to define a hyperbolic separation space around the normal data points. Any point that falls into the separation space is considered normal, while points that lie outside the separation space are considered anomalies. OCSVM models are typically trained with a training data set that contains only positive examples (normal data points). The model then learns to define a parting surface that corresponds to normal data points, while separating anomalies from normal data points. The model can then be used to classify new data sets by checking whether or not they are within the defined separation space.
Isolation Forests: Isolation Forests are a decision tree-based model that can be used to detect anomalies in large and complex datasets. The model separates the normal data points from the anomalies by creating a tree of decision rules. Anomalies can be identified by a shallow depth in the tree because they deviate from the normal data density faster than normal data points.
Local Outlier Factor (LOF): LOF is a clustering-based model used to detect anomalies in data-dependent spaces. The model calculates the local densities of the data points and identifies anomalies that lie in areas of low local density.
Support Vector Data Description (SVDD): SVDD is an SVM-based model used to detect anomalies in data. Unlike OCSVMs, SVDD attempts to define a hyperbolic separation space around the normal data points instead of separating them from anomalies. Anomalies are then considered as data points that lie outside the defined separation space.

The one-dimensional vector can optionally be processed before the machine learning analysis (data pre-processing), wherein the processing can include at least one of the following operations:
Noise suppression
Feature Extraction: Feature Extraction or feature selection is the process of extracting from the 1D vector relevant features that are relevant to the machine learning model. This can be done by statistical methods such as calculating averages, variances, or correlations, or by more advanced methods such as wavelet transformations or Mel Frequency Cepstral Coefficients (MFCCs) for audio data.
Preprocessing: Preprocessing refers to the process of normalizing, scaling, or otherwise preparing the 1D vector to make it suitable for the machine learning model. This can be done by standardization or min-max scaling to bring the data to a common scale, or by normalization to bring the data to a specific distribution.
Data/Dimensionality Reduction: Data reduction refers to the process of reducing the dimensionality of the 1D vector to reduce processing complexity or improve the accuracy of the machine learning model. This can be done by Principal Component Analysis (PCA) or other dimension reduction methods.

These processing operations (data pre-processing) can be applied individually or in combination.

In an advantageous embodiment, the dimensionality reduction and/or the feature selection of the at least one 1D vector comprises at least one processing method from a group of processing methods comprising the group of processing methods: principal component analysis, T-distributed stochastic neighbor embedding, linear discriminant analysis, truncated singular value decomposition, uniform manifold approximation and projection, independent component analysis, sparse representation, partial least squares regression and kernel principal component analysis.

At least one scatterplot and/or at least one one-dimensional vector is used for predicting the blood age. The scatterplot is analyzed by at least one deep learning model, while the one-dimensional vector is processed by at least one machine learning model. The method makes it possible to predict the state precisely and effectively in vitro.

If at least two deep learning models and/or at least two machine learning models and/or deep learning models are used in combination with one machine learning model, this is called the prediction of the state by an ensemble. The idea behind the ensemble is that different models can have different weaknesses and strengths, and that the combination of several models can compensate for the weaknesses and reinforce the strengths. An ensemble prediction is made by combining the predictions of several individual models. Ensemble technology can be used to improve the predictive accuracy and robustness of artificial intelligence by combining different models or model instances to make a consolidated prediction of the state. The exact method of prediction depends on the specific ensemble technique used. Techniques may include one or more of the following:
Bagging: Bagging creates multiple copies of the same model with different training data sets. If the ensemble is to make a prediction, the predictions of each individual model are combined by averaging them. The idea is that combining multiple copies of the same model with different sets of training data improves predictive performance by reducing variability.
Boosting: In boosting, several weak models are trained in succession. Each model attempts to correct the errors of the previous model by focusing on the incorrectly predicted examples. If the ensemble is to make a prediction, the predictions of each individual model are combined by weighing them. The idea is that combining multiple weak models will result in a strong model by correcting the errors and reinforcing the strengths.
Stacking: Stacking trains several different models and uses the output of each model as input for a parent model that makes a final prediction. If the ensemble is to make a prediction, the predictions of each model are combined by using them as input to the parent model. The idea is that combining several different models that focus on different features will result in better predictive performance.
Hard voting: The predictions of several individual models are combined and the final prediction is decided by a majority vote. If the ensemble is to make a prediction, the class predicted by the majority of classifiers is selected. Hard voting is a simple and effective way to improve the predictive performance of classifiers by reducing variance and reducing overfitting. It can also be used in combination with other ensemble techniques such as bagging or boosting.
Soft voting: Soft voting aggregates and weights the predictive probabilities of each model to produce a final predictive probability for each class. The weighting can be adjusted manually or automatically based on the prediction performance of each classifier. The final prediction is then made using the class with the highest probability of prediction. The idea behind soft voting is that models that have higher predictive accuracy should be weighted more than those that have lower predictive accuracy. Soft voting is particularly useful when the models in an ensemble have different strengths and weaknesses or when the decision boundary between classes is blurred.
Random subspace
Mixture of experts
Bayesian model averaging

Prediction of blood age includes class designation and/or predictive probability.

In an advantageous embodiment, in vitro and/or ex vivo states are included which are based on processes outside a living organism, including changes in the morphology of the blood cells, cell composition, cell function or other features of the individual cells as a result of storage, handling and/or analysis.

In an advantageous embodiment, in vivo states are based on processes within a living organism, including physiological and pathological states such as diseases, biological age, pregnancy, drug action, state of health, nutritional deficiency, hereditary disorders, dehydration, blood clotting disorders, infections and/or anemia.

In an advantageous embodiment, post-mortem states are based on processes of a dead organism, including changes in morphology, cell composition, cell function or other characteristics of the individual cells as a result of diseases, presence of drugs, health status before death, drugs, poisons and/or toxic substances, as well as changes caused by the decay and autolysis of cells and tissues after death.

In an advantageous embodiment, at least one result report is produced by a computer device, wherein the result report comprises a graphic and/or an information text and/or a probability and/or a score and/or a class for at least one state, wherein the presentation of the result report comprises the presentation on the computer device, for example a PC or a portable computer and/or a mobile terminal and/or laboratory device.

In an advantageous embodiment, all described models are trained and/or validated on the basis of a prefabricated database, wherein the database comprises measured blood parameters and/or scatterplots, wherein the database can be extended with new measured blood parameters and/or scatterplots in order to continuously improve the performance and accuracy of the models, wherein the database can comprise information about known states, diseases or other relevant information which contributes to better interpretation and analysis of the measured blood parameters and/or scatterplots. The training methods for the aforementioned models of artificial intelligence can comprise both supervised and unsupervised learning, wherein supervised learning focuses on the use of annotated data in the database for identifying patterns and contexts, while unsupervised learning allows the recognition of patterns and contexts in the data without prior annotation in order to identify novel findings and possibly previously unknown states or diseases. These artificial intelligence models can be trained using ensemble learning techniques that combine multiple models or algorithms to increase the performance and accuracy of predictions and compensate for the weaknesses of individual models or algorithms.

In an advantageous embodiment, the artificial intelligence is capable of continuously optimizing and adapting the method on the basis of new data and findings in order to improve the predictive accuracy and robustness by learning from its own feedback and the errors.

In an advantageous embodiment, a user interface is additionally provided which makes it possible to facilitate the input of parameters and/or scatterplots and the display of the results and reports for medical personnel and/or patients.

In an advantageous embodiment, the method allows integration and use of external data sources, including clinical data, demographic information, medical history and/or genetic data, in order to provide additional context and improved predictive accuracy in the determination of states.

In an advantageous embodiment, the method offers the possibility of introducing human expertise and feedback into the training and validation process of artificial intelligence in order to increase the model accuracy and reliability, in particular in cases where the amount of data is limited or incomplete.

In an advantageous embodiment, the method makes it possible to produce personalized reports which are tailored to the individual needs and requirements of medical personnel and/or patients, in that the presentation, the information content and the format of the reports can be adapted.

In an advantageous embodiment, the method comprises supplying real-time blood parameter data from the hematology analyzer in order to carry out continuous monitoring and real-time analysis of states.

In an advantageous embodiment, the method comprises training the at least one deep learning model and/or the at least one machine learning model, wherein the training comprises monitored and/or unsupervised learning, wherein supervised learning comprises the use of annotated data in a database for identifying patterns and contexts, while the unsupervised learning permits a recognition of patterns and contexts in the data of the database without prior annotation in order to identify novel findings and possibly hitherto unknown states or diseases.

In an advantageous embodiment, the training includes transfer learning, in which pre-trained models from related domains or applications are used as a starting point for the training and an adaptation to the specific blood parameters and/or scatterplots, in order to increase the efficiency and effectiveness of the training and to reduce the required amount of training data.

In an advantageous embodiment, the training comprises active learning in which the at least one deep learning model and/or the at least one machine learning model specifically search for examples in the database which can improve their performance and accuracy the most.

In an advantageous embodiment, the training includes the recording of inputs from a user for annotation and/or confirmation of the examples in order to optimize the training process.

In an advantageous embodiment, the training comprises at least one ensemble learning method in which a plurality of learning methods are combined in order to increase the performance and accuracy of the predictions and to compensate for the weaknesses of individual models or algorithms.

In an advantageous embodiment, the training comprises incremental learning, in which the deep learning model and/or the machine learning model are continuously and stepwise updated on the basis of newly added blood parameters and/or scatterplots in the database in order to improve the performance and accuracy of the models over time and to be able to react to changes in the underlying data.

In an advantageous embodiment, the method executes at least one data augmentation process in order to increase the size and diversity of the training data in the database, in order to reduce the risk of overfitting and to improve the performance and accuracy of the models mentioned.

In an advantageous embodiment, the at least one data augmentation process has a synthetic generation of blood parameters and/or scatterplots which are based on existing data, stochastic methods, statistical models or artificial intelligence algorithms being used in order to generate realistic and representative data for the training of the models.

In an advantageous embodiment, the at least one data augmentation process has at least one transformation of existing blood parameters and/or scatterplots, wherein the at least one transformation has rotations, scales, reflections, shearings, noise and/or distortions, in order to increase the diversity of the training data and to increase the robustness of the determination of the at least one state. Noise can be understood as adding numerical noise.

In an advantageous embodiment, the at least one data augmentation process has a combination of blood parameters and/or scatterplots from different sources, such as, for example, different devices, techniques, patient populations or clinical studies. This can improve the representativeness of the training data for a broader application of the artificial intelligence models.

In an advantageous embodiment, the at least one deep learning model and/or the at least one machine learning model is designed in such a way that it adapts the degree of data augmentation on the basis of boundary conditions, for example factors such as the size of the existing database, the number of previous training iterations and/or the current performance and accuracy of the relevant model, in order to improve the efficiency of the training and to make the training process more efficient.

In addition to blood age, the method can be used to determine other in vitro and in vivo states, including:
Anemia: Deficiency of red blood cells or hemoglobin in the blood can affect the size, shape, and number of red blood cells.
Infections: Infections can change the number and type of white blood cells in the blood.
Inflammation: Inflammation can also affect the number and type of white blood cells, particularly the number of neutrophils.
Blood clotting disorders: Disorders that interfere with the function of platelets or clotting factors in the blood can affect blood clotting and the number and function of platelets.
Dehydration: Dehydration may increase hematocrit and increase red blood cell levels in the blood.
Bone marrow disorders: Bone marrow disorders such as leukemia and myelodysplastic syndromes can affect the production of red and white blood cells in the bone marrow and produce abnormally shaped blood cells.
Hereditary disorders: Some hereditary disorders, such as sickle cell anemia and thalassemia, can affect the size and shape of red blood cells.
Cancer: Cancers such as lymphomas and leukemias can affect blood counts by changing the number and function of blood cells in the blood.
Autoimmune disorders: Autoimmune disorders such as systemic lupus erythematosus and rheumatoid arthritis can affect the number and type of white blood cells.
Immune system disorders: Immune system disorders such as HIV/AIDS can affect the number and function of white blood cells and increase the risk of infections.
Kidney disorders: Kidney disorders can affect blood counts by interfering with the production of erythropoietin, a hormone that causes red blood cells to be produced in the bone marrow.
Nutritional deficiencies: Nutritional deficiencies, particularly deficiencies of iron, vitamin B12, or folate, can affect blood counts and lead to anemia.
Drugs: Some drugs can affect blood counts by inhibiting the production of blood cells in the bone marrow or by shortening the life of blood cells.
Pregnancy: During pregnancy, the number of white blood cells in a woman's blood may be increased, while the number of red blood cells may be decreased due to increased plasma uptake in a woman's body.
Genetic disorders: Certain genetic disorders, such as sickle cell disease and thalassemia, can affect the size and shape of red blood cells and cause anemia.
Liver disorders: Liver disorders, such as cirrhosis of the liver, can affect the number and type of blood cells because the liver plays an important role in the production of blood cells.
Arteriosclerosis: A disorder in which fat and other substances are deposited in the walls of arteries. This may reduce blood flow and lead to an increased number of white blood cells in the blood.
Coronary artery disease: A disease in which plaque accumulates in the coronary arteries, which can reduce blood flow to the heart. This can cause the heart to receive less oxygen and increase the number of red blood cells in the blood.
Heart attack: A heart attack occurs when blood flow to the heart muscle is suddenly blocked, usually by a blood clot. This can lead to damage to the heart muscle and affect the number of blood cells in the blood.
Heart failure: A disorder in which the heart is no longer able to pump enough blood to provide the body with oxygen and nutrients. This can lead to an increase in the number of white blood cells and to impairment of red blood cells.
Blood age after blood sampling: After blood samples are taken, various factors, such as temperature, storage states, and type of treatment, can change the properties of blood cells. Blood cells, especially red blood cells, may age over time and change their functions and characteristics. The breakdown of blood cells can be caused by bacteria or enzymes that are present in the blood or can be added during storage. Changes in cell membranes: The cell membranes of blood cells can be damaged by exposure to chemicals or physical forces.
Biological age: As people age, the number of white blood cells in the blood may decrease, while the number of red blood cells and platelets may increase. The type of blood cells may also change, e.g. the size and shape of red blood cells may change.

The invention further relates to a system for determining states in vivo and in vitro by analyzing blood parameters measured in a hematology analyzer, comprising a computer device having a computing unit, a memory unit connected thereto and an input unit, wherein the system is designed for
a) detecting blood parameters measured in a hematology analyzer, through the input unit, wherein the blood parameters comprise quantitative and qualitative measurement variables, wherein the measurement variables comprise the properties of individual cells, wherein the individual cells comprise white blood cells, red blood cells and platelets, wherein the white blood cells comprise monocytes, lymphocytes, basophils, eosinophils and neutrophils;
b) producing at least one scatterplot by the arithmetic unit, each axis of the scatterplot comprising a different measurement variable;
c) determining at least one in vivo and/or in vitro and/or post-mortem state by means of the computing unit by means of at least one deep learning model and/or a machine learning model, wherein the at least one deep learning model receives at least one scatterplot image as input variable, wherein the at least one machine learning model contains at least one 1D vector as input variable; and
d) generating a report automatically by the computing unit, wherein the report comprises at least one result on the determination of the at least one state.

In an advantageous embodiment, the system has a communication interface for transmitting results and reports to other computer systems, laboratory information systems (LIS), hospital information systems (HIS) and/or electronic patient records (EPA).

The system is furthermore preferably designed to carry out at least one or more of the above-described method steps.

### Exemplary Embodiment: Age of the Blood

This is the first demonstration of how deep learning models can be used to accurately determine the age of in vitro blood samples after collection, based on the properties of blood cells measured by a hematology analyzer.

A total of 228 venous blood samples were measured on the hematology analyzer. 149 of 228 blood samples were measured during the first two hours after blood collection and 79 of 228 blood samples were measured 24 hours after blood collection. All measurements were performed on the Abbott Cell-Dyn Ruby hematology analyzer. The measurements were transferred from the hematology analyzer to the laboratory information system (LIS) according to the ASTM protocol and downloaded and stored as TEXT files (FIG. 7).

According to the instructions in the Cell-Dyn Ruby System Host Interface Specification (LIST NO. 09H05-01 Revision C), the inventors of the patent were able to decrypt the encrypted information in the TEXT files using a self-written algorithm in Python and store it as 228 Excel tables in a folder called database. 149 tables with the measurement data of the 2-hour blood sample were stored in one folder and 79 tables with the measurement data of the 24-hour blood sample were stored in the other folder (FIG. 8).

Each table consisted of 7 columns and 2000 rows (FIG. 9), wherein:
The 1st Column contained measurements from the ALL (Axial Light Loss) channel representing cell size or light scattering signals below 0°,
The 2nd Column contained measurements from the Intermediate Angle Scatter (IAS) channel representing cellular complexity or light scattering signals below 10°,
The 3rd Column contained measurements from the polarized side scatter (PSS) channel representing the nuclear lobularity of the cells, or light scattering signals below 90°,
The 4th Column contained measurements from the Depolarized Side Scatter (DSS) channel representing the granularity of the eosinophils, and
Columns 5 to 7 contained measurements of red blood cell and platelet properties.

This database was randomly divided into three parts (FIG. 10): training, validation and test data set. The training data set was used to train a deep learning model. The model was trained on the training data set to learn how to convert certain inputs into certain outputs. During training, the model iteratively traversed the training data and adjusted its internal parameters to match the outputs as closely as possible to the expected outputs (<2 h=0, 24 h=1). The validation data set was used to monitor the performance of the deep learning model during the training process after each training period and to avoid overfitting the model. Overfitting occurs when the model fits the training data too well, but performs poorly in predicting new data. The validation data set consists of data that is not used for training the model, but only for monitoring the model during training. After training, the model was tested on an independent test data set to assess its performance. If the model produces good results on the test data, it can be used to make predictions on new, previously unknown data.

A convolutional neural network (CNN) in Keras was chosen as the deep learning model (FIG. 11). Keras is an open source library for deep learning in Python. It is designed to facilitate the development of deep learning models through a simple, intuitive and modular interface. Keras offers a variety of ready-made layers and models that allow developers to quickly and easily build and train complex models and can be used for both scientific and commercial purposes. Designed to be simple and easy to use, Keras provides a quick and effective way to create and train deep learning models without having to worry about the details of implementation. It supports both CPU and GPU calculation and can be run on various platforms such as Windows, Linux and MacOS. Keras is licensed under the MIT license. The MIT license is a permissive, open-source software license that allows users to use, copy, modify, and distribute the code for various purposes as long as the license text and copyright notice are retained. The MIT license has few limitations, making it business-friendly by allowing the software to be used and customized in commercial and proprietary projects.

The deep learning model that was trained consisted of several layers, each performing specific operations to convert the input image (scatterplot) into an output. Here is a brief explanation of each layer:
Conv2D: A convolutional layer that applies a fixed number of filters to the input image and extracts the characteristics of the image.
Batch Normalization: A normalization layer that normalizes the activations in the previous layer to reduce gradient explosion and disappearance and accelerate convergence.
MaxPooling2D: A pooling layer that reduces the output image size by extracting the maximum value in each pooling window.
Flatten: A layer that converts the 2D output image into a 1D vector to forward it to a fully connected layer.
Dropout: A layer that randomly zeroes some of the previous layer's output elements to reduce overfitting.
Dense: A fully connected layer that transforms the activations of the previous layer into a new space.
Activation: A layer that activates the output of the previous layer through an activation function such as ReLU or Sigmoid.

The last Dense layer had only one output node with the sigmoid activation function, since this is a binary classification problem (<2 h=0, 24 h=1). The model was trained with a binary cross entropy loss and the Adam optimizer.

As an input variable for Deep Learning models, 256×256 2D scatterplots from the ALL channel measurements (1. Column in the Excel tables) and IAS channel measurements (2. Column in the Excel tables) were used during training, validation and testing (FIG. 12).

After training, the model was tested on an independent test data set to assess its performance. AUROC was chosen as the performance metric. AUROC stands for “Area Under the Receiver Operating Characteristic Curve” and is a metric for evaluating the performance of a binary classifier, e.g. a deep learning model, which distinguishes between two classes. The Receiver Operating Characteristic (ROC) curve is a graphical representation of how well a classifier is able to tell classes apart by plotting the true positive rate (TPR) against the false positive rate (FPR). The area under the ROC curve (AUROC) indicates how well the overall classifier performs and is a measure of the model's performance. A perfect prediction would have an AUROC of 1, while a random prediction would have an AUROC of 0.5. A higher AUROC means that the classifier achieves better separation between classes and thus has higher performance. AUROC is an important metric for assessing the performance of deep learning models, especially in classifying medical images, making diagnoses or predicting diseases.

The following figure shows the ROC curve and AUC obtained on the test data set. AUROC is 0.96 (FIG. 13).

An AUROC of 0.96 indicates that in most cases the model has a very high probability of making a correct prediction in the future. At a threshold value of 0.788, the following results were obtained on the test data set (FIG. 14).

The Confusion Matrix shows that all 46 blood samples not older than 2 hours have been correctly recognized. Thus, the specificity is 100.00%. In this example, specificity indicates how well the model identifies blood samples that are 2 hours old. Out of 21 blood samples taken after 24 hours, 20 blood samples were correctly determined. Thus, the sensitivity is 95.24%. Sensitivity indicates how well the model actually identifies blood samples that are 24 hours old. Sensitivity and specificity are important metrics for evaluating the performance of a classification model.

However, as a high AUROC was achieved on a test data set with only 67 data, the results were interpreted with caution, as a small test data set may not be representative of the total data set. In this case, a high AUROC could also be the result of randomness. It was decided to perform a 4 fold cross-validation. The use of cross-validation methods such as the 4-fold cross-validation method is used to assess the performance of a deep learning model and to ensure that the model can generalize well not only to the specific data but also to other data. In the 4-fold cross-validation method, the data set is divided into four subsets of approximately equal size, each subset being used once as a test data set and three times as a training data set. This division trains and tests the model on various combinations of training and test data sets, improving the robustness of the results. In a 4-fold cross-validation method, the AUROC is calculated for each test data set and the average AUROC across all test data sets is used as a measure of the performance of the model. Using a 4-fold cross-validation method can improve the robustness of the results because they are based on a larger set of test data sets. The following figure shows AUROCs for all 4 test folds, the average for AUROC being 93.13% (FIG. 15).

A mean AUROC of 0.93 after 4 fold validation means that the deep learning model still shows very good to excellent performance in binary classification. An AUROC of 0.93 is close to the ideal performance of a binary classifier (AUROC=1), indicating that the model is able to distinguish the two classes (2 h blood vs. 24 h blood) very well. Such a high degree of accuracy is particularly important in medical applications where it is important to determine a clinical state with high certainty or to make a prediction with high reliability.

The solution described in the example for predicting the age of in vitro blood samples using deep learning models has the potential to be used in different areas. Some possible applications are:
Medical research: Predicting blood age can be important in studying blood disorders, blood cell aging, and the effects of drugs or treatments on the blood.
Transfusion medicine: When transfusing blood, it is important to ensure the freshness of the blood. This solution could help to monitor the quality of blood supplies and ensure that they are used within the appropriate timeframe.
Forensics: In forensic science, determining the age of blood samples could help determine the timing of crimes or accidents more accurately.
Biotechnology and the pharmaceutical industry: The technology can be used in the development of new drugs or therapeutic approaches by helping to deepen the understanding of the effects of substances on blood and blood cells.

It is important to note that the performance and applicability of this solution in practice will depend on the quality and robustness of the trained model. To ensure that the model is effective and reliable, it can be tested on larger and more diverse datasets in clinical trials and adjusted as necessary.

### Exemplary Embodiment: Suspected Acute Coronary Syndrome

A 36-year-old patient entered the admission department with a preliminary diagnosis of “Arteriosclerotic heart disease, Acute Coronary Syndrome without ST reversal. Acute heart failure. Killip Class I” one or two hours after a typical pain syndrome. The electrocardiogra examination was performed in the receiving station, which also showed no increase of the ST segment on the electrocardiogram. Venous blood samples were then collected for laboratory testing. Blood samples collected outside the method were made available for testing, which included the highly sensitive cardiac troponin I method and clinical blood analysis. The results of laboratory studies were: urea 4.2 mmol/L (3.0-9.2); ALT 16 units/L (0-55); AST 12 units/L (5-34); total protein 70 g/L (64-83); creatinine 74 μmol/L (64-111); total bilirubin 6.2 μmol/L (3.4-20). 5); glucose 7.5 mmol/L (3.9-5.5); potassium 3.7 mmol/L (3.5-5.1); sodium 137 mmol/L (135-145); calcium ionized 1.23 mmol/L (1.13-1.32); APTV 78.7 s (25.1-36.5); MNO 0.97 (0.2-0.5). 90-1.20); prothrombin 118.0% (70.0-140.0); prothrombin time 11.0 s (9.4-12.5); leukocytes 12.4 10E9/L (4.0-9.0); (NEUT) neutrophils 10.0*109/L (2.0-5.5); (NEUT %) neutrophils 80.0%
(48.0-78.0); (LYM) lymphocytes 1.79*109/L (1.20-3.00); (LYM %) lymphocytes 14.3%
(19.0-37.0); (MON) monocytes 0.57 109/L (0.09-0.60); (MON %) monocytes 4.6% (3.0-11.0); (EOS) eosinophils 0.07*109/L (0.00-0.30); (EOS %) eosinophils 0.52% (1.00-5.00); (BAS) basophils 0.07*109/L (0.00-0.06); (BAS %) Basophils 0.52% (0.00-1.00); (HGB) Hemoglobin 134 g/L (130-160); (HCT) Haematocrit 40.7% (40.0-48). 0); (RBC) erythrocytes 4.45 10*12/L (4.00-5.60); (MCH) Mean red blood cell hemoglobin 30.1 pg (24.0-34.0); (MCHC)
mean hemoglobin concentration in blood cells 32.9 g/dL (30.0-38.0); (MCV) mean erythrocytes volume 91.4 fL (75.0-95.0); (RDW-CV) erythrocytes distribution by 11.0% (11.5-16.0%). 5); (PLT) platelets 262*109/L (180-400); (MPV) mean platelet volume 11.5 fl (7.4-10.4);
cardiac troponin I (high sensitivity method) 37.9 ng/ml (upper reference limit 26.2 ng/ml).

After the blood was measured, the raw measurement data was transferred to the PC. Subsequently, two scatterplots for the trained deep learning models were generated and combined to form a global scatterplot. In parallel, the global vector with 4216 elements was derived:

$${V}_{GLOB} = {\{ \begin{matrix}\{ {V}_{Neutrophils} \} \{ {V}_{Lymphocytes} \} \\ \{ {V}_{Monocytes} \}\end{matrix} \}}_{4216}$$

wherein 4216 elements represent the measured properties of the leukocyte subpopulations. The global vector is standardized by deleting the mean and scaling it to a single dispersion based on a predefined database:

$${V}_{GLOB}^{STAND} = \frac{\{ {V}_{GLOB} \} - {\{ and \}}_{database}}{{\{ s \}}_{database}}$$

wherein the vector {u} represents the means of the global vector property elements in the prefabricated database and the vector {s} represents the standard deviations of the global vector elements in the prefabricated database.

After standardization, a PCA was used as an example to reduce the dimensionality of features from 4216 elements to 4 elements, which are referred to as main components, and at the same time to obtain the greatest possible variability (information) of the features. After applying the principal component analysis, all 4216 elements of the standardized global vector were reduced to the vector in the 4-dimensional subspace:

$${V}_{GLOB}^{STAND \& RED} = \{ \begin{matrix}\begin{matrix}- 40.48 58.66 \\ - 5.4\end{matrix} \\ 0.75\end{matrix} \}$$

At first glance, it seems difficult to extract information for patient diagnosis from the values of this reduced vector. For this purpose, an ensemble of the ensembles of the trained models of machine learning is used. The ensemble consists of an ensemble of Artificial Neural Networks, an ensemble of K-Nearest Neighbors models, an ensemble of Random Forest models, an ensemble of AdaBoost models, an ensemble of Gradient Tree Boosting models and an ensemble of Support Vector Machines models, with the individual ensembles each trained on the pre-built database. The standardized and reduced global vector is used as the input vector for all ensembles, while the global scatterplot is used for a deep learning model. In the above case, the votes for AKS are counted for individual ensembles (hard voting):

| | Ensemble from | Positive (AKS) | Negative (no AKS) |
|---|---|---|---|
| 1 | K-Nearest Neighbors | YES | No |
| 2 | Neural networks | YES | No |
| 3 | Random forest | YES | No |
| 4 | Gradient boosting | YES | No |
| 5 | AdaBoost | YES | No |
| 6 | Support Vector Machines | YES | No |
| 7 | Deep Learning CNN | YES | No |

The final result for AKS after the hard voting procedure is positive. The decision was made to perform percutaneous coronary intervention. The patient underwent coronary angiography followed by transluminal dilation and stenting of the infarct-dependent coronary artery.

CORONAROGRAPHY No 7175 of 07.06.2018: Left type of blood circulation. Left coronary artery: Barrel—without stenosis. Anterior ventricular branch—mouth stenosis 5-50%, middle third subocclusion. A. intermedia—stenosis in the proximal third of 90%. Diagonal branches—without stenosis. Bending branch (BB)—without stenosis. Blunt—edged branches-without stenoses. Right coronary artery: hypoplassed. Acute Edge Branch: No stenosis. Rear branch (ROB)—without stenoses. Rear interventricular branch—without stenosis.

ORONAROPLASTY AND PMV STENTING No 7176 of 07.06.18: stenosis zone of PMV (average third) BC 2.0*20.0 mm, p=18 atm. The stent with a drug coating of 2.75*33.0 mm is implanted in the middle third of the BC 2.0*20.0 mm, p=16 atm. Control: TIMI III degree blood flow. No infiltration shadows were observed on chest x-rays in 2 projections dated 8 Jun. 2018. The roots are structural, not enlarged, the left one is partially blocked. The lung pattern was not altered. The diaphragm is contoured. Heartshadows without facial features. Sinus is free.

The following treatment was performed: beta-blockers, anticoagulants, double disaggregation therapy, statins (the dose of Crestor was reduced by 20->10 mg/day due to the increase in transaminase levels), gastroprotectors. The patient refused rehabilitation treatment in the sanatorium.

In the postoperative period, the maximum concentration of cardiac troponin I in dynamic observation reached 7522.5 ng/ml. The hospital stay lasted 12 days. The final diagnosis was arteriosclerotic heart disease. Acute myocardial infarction of the anterior parietal region, high lateral proportions of the left ventricle without elevation of the ST segment of 07.06.18. Coronary plastic surgery and stenting of 07.06.18″. The patient was discharged on 19.06.2018 for further ambulatory observation at his place of residence.

### BRIEF DESCRIPTION OF THE FIGURES

The figures show:

FIG. 1 measurement technology of a hematology analyzer;

FIG. 2 measurement technology of a further hematology analyzer;

FIG. 3 measurement technology of a further hematology analyzer;

FIG. 4 prediction of a state using an ensemble approach;

FIG. 5 procedure of the method in a laboratory environment;

FIG. 6 steps of the computer-implemented method;

FIG. 7 measurement data;

FIG. 8 creation of a database from measurement data;

FIG. 9 measurement data;

FIG. 10 division of measurement data into training, validation and test data sets;

FIG. 11 architecture of a deep learning model for determining the blood age;

FIG. 12 scatterplot;

FIG. 13 AUROC curve;

FIG. 14 metrics for test data set in determining blood age; and

FIG. 15 four AUROC curves.

FIG. 1 illustrates by way of example the measurement technology used in Sysmex hematology analyzers (https://www.sysmex.com). Sysmex has developed an innovative fluorescence flow cytometry technology that provides detailed information on cell size, cell structure and cell contents. In flow cytometry, cells and particles are analyzed by passing them through a very narrow flow cell. The process begins with the collection of a blood sample, which is then dosed and diluted to a fixed ratio. The sample is then labeled with a special fluorescent marker developed by Sysmex, which binds specifically to nucleic acids. In the next step, the labeled sample is transported into the flow cell. During the analysis, the sample is illuminated with a semiconductor laser beam, which makes it possible to distinguish the cells on the basis of three different signals: 1) Forward Scattered Light (FSL): This signal is proportional to the size of the cell and allows conclusions to be drawn about the cell size. 2) Side scattered light (SSL): This signal provides information about the inner cell structure and complexity, as it is related to the granularity and structural properties of the cell. 3) Side Fluorescent light (SFL): This signal detects the RNA/DNA content of the cell by measuring the fluorescence intensity produced by the fluorescent marker bound to nucleic acids. By combining these three signals, Sysmex's FFC technology enables precise analysis and differentiation of cells in blood samples.

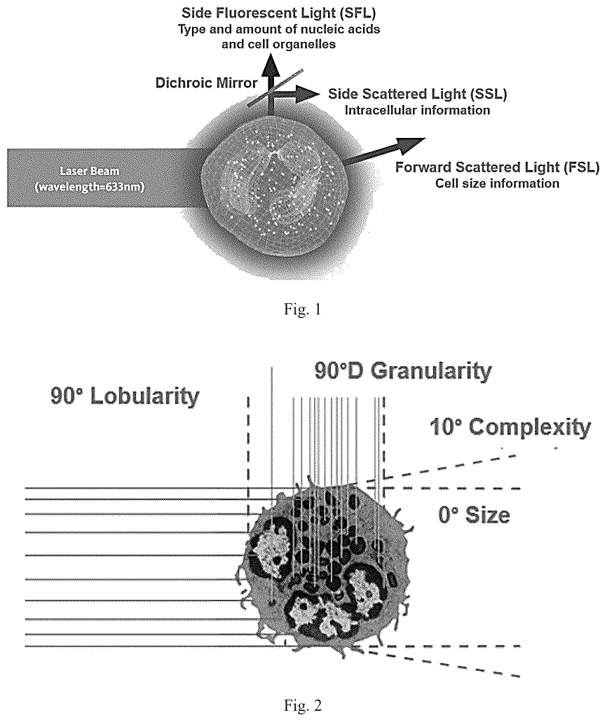

FIG. 2 illustrates by way of example the measuring technology of Abbott (https://www.abbott.com/). Abbott has developed the innovative and patented Multi Angle Polarized Scatter Separation (MAPSS™) technology that delivers accurate first pass WBC and differential results by laser measurement of up to 20,000 cells and four angles of optical analysis from a single dilution. MAPSS™ technology uses four light scattering detectors to determine different cellular properties. A special feature of this method is the use of a depolarized light detector, which enables the specific identification of eosinophilic granulocytes. The four detectors generate the following signals: 0° or Axial Light Loss (ALL): This signal is related to the size of the cell and allows conclusions to be drawn about the cell size. 0° to 10° Intermediate Angle Scatter (IAS): This signal depends on the cellular complexity and provides information about the inner cell structure.

90° polarized side scattering (PSS): This signal refers to the core lobularity or segmentation of the cell and allows the identification of cellular structural features. 90° depolarized side scatter (DSS): This signal is related to eosinophils granules and allows the specific recognition of eosinophilic granulocytes in the sample.

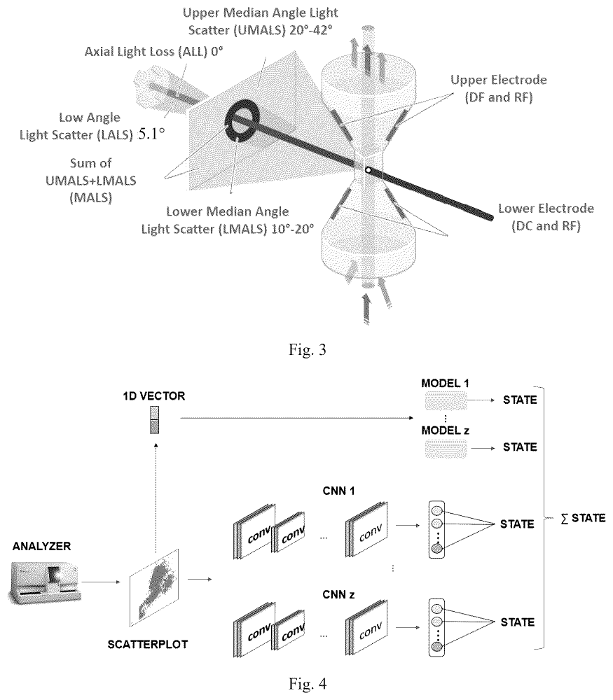

FIG. 3 illustrates by example the measuring technology of Beckman Coulter (https://www.beckmancoulter.com/). For example, the DxH 800/DxH 600 devices have the Multi-Transducer Module (MTM), which measures several angles of light scattering. The MTM flow cell detects the volume, conductivity, multiple angles of light scattering and axial light loss: Lower electrode (DC and RF>conductivity): This electrode measures the conductivity of the cells, which is detected with direct and alternating current. Upper electrode (DC and RF>conductivity): Similar to the lower electrode, this electrode also measures the conductivity of the cells, which is detected with direct and alternating current. Axial light loss (ALL) 0°: This signal is related to the cell size and allows conclusions to be drawn about the size of the cell. Low-angle light scatter (LALS) 5.1°: This angle of light scattering provides information about cellular granularity and structure. Light scatter in the lower median angle (LMALS) 10°-20°: This range of light scattering allows a more accurate determination of cell structure and complexity. Light scatter in the upper median angle (UMALS) 20°-42°: This range of light scattering allows an even more precise analysis of the cell structure and complexity. The fifth light scattering channel is the sum of the UMALS and LMALS regions (MALS): The combination of the information from both median angle regions allows a more comprehensive analysis of the cell properties.

FIG. 4 shows by example the prediction of a state with the aid of an ensemble approach. A scatterplot is created from the blood parameters obtained from a hematology analyzer. This diagram is used as an input variable for deep learning models such as Convolutional Neural Networks (CNNs). At the same time, a one-dimensional (1D) vector is generated from the scatterplot by a flattening method. This 1D vector is used as an input variable for machine learning models that can process one-dimensional structure data. All models predict the patient's state. The final determination of the state is based on a soft-voting or hard-voting method, such as, for example, the weighted average of the probabilities for the presence of a specific state. By combining different models and approaches in one ensemble, the strengths of each model can be exploited and weaknesses can be compensated. This leads to improved predictive accuracy and contributes to the effective diagnosis and monitoring of hematological diseases.

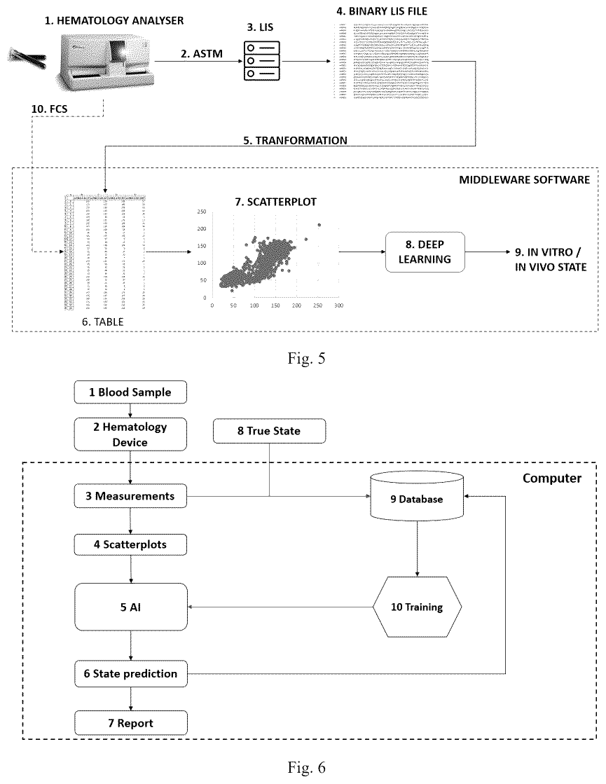

FIG. 5 shows, by way of example, the procedure of the method in a laboratory environment. The measurement data are taken from the Abbott Cell-Dyn Ruby 1 brand hematology analyzer and transmitted to the Laboratory Information System (LIS) 3 in ASCII format in accordance with ASTM Protocol 2. The blood parameters for the compilation of the scatterplots are provided in binary format 4. The blood parameters are then decrypted according to the Cell-Dyn Ruby System Host Interface Specification (LIST NO. 09H05-01 Revision C) 5 and stored in an Excel spreadsheet 6. A scatterplot 7 is created from the columns of this Excel table, which serves as an input variable for the deep learning model 8. Based on the scatterplot, the deep learning model makes a prediction for the patient's state. The integration of modern analytical equipment, standardized protocols and AI-based models in a laboratory environment can achieve efficient and reliable diagnosis and monitoring of hematological diseases.

In the exemplary illustration of FIG. 6, all steps of the computer-implemented method are summarized. A blood sample taken before the method is provided for examination in the method according to the invention, which is referred to here as step (1). In step (2), the quantitative and qualitative properties of the individual cells of the blood sample are measured in an analyzer. In step (3), the measurement data are transmitted to the computer. In step (4), the scatterplot is created. In step (5), the scatterplot is used as an input variable for the deep learning model. In parallel, a 1D vector can be derived from the scatterplot and used as an input variable for the machine learning models. In step (6), a state (e.g. blood age in vitro or a disease in vivo) is determined. In step (7), the diagnosis is output on a graphical interface or in the form of an automatically generated report. In step (9), the predicted diagnosis together with corresponding measurement data from (3) can be stored in the prepared database in order to expand the database with a new case and to train new models according to the semi-supervised approach. The prepared database can also be extended with measurement data and the corresponding true diagnoses in step (8). The purpose of the database is to train and evaluate the models. The models in step (5) can be replaced at any time by the newly trained models in step (10), as long as the newly trained models achieve better results on the test data set.

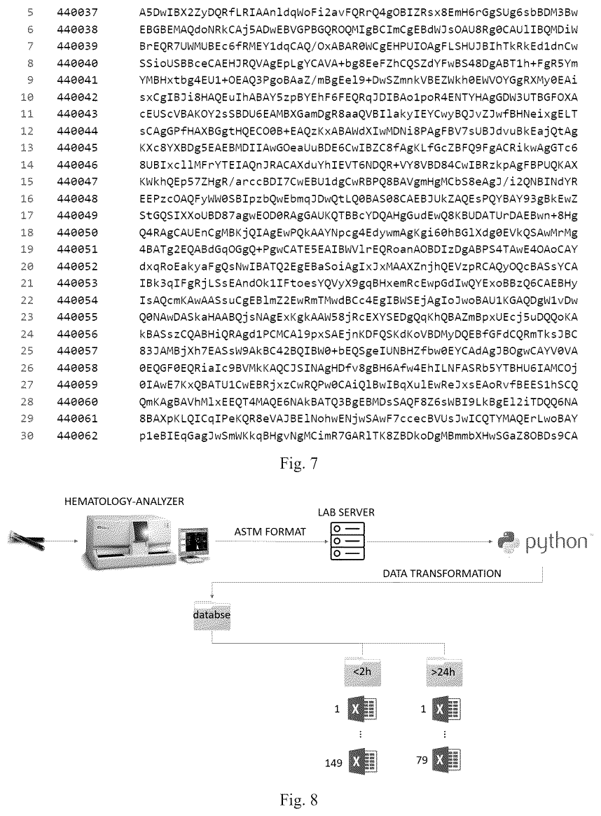

FIG. 7 shows, by way of example, the measurement data which were transmitted from the hematology analyzer to the laboratory information system (LIS) in accordance with the ASTM protocol. This data was downloaded and saved as text files. This includes the blood parameters for the creation of the scatterplots in binary format. The use of standardized protocols such as the ASTM protocol enables reliable and consistent transmission of measurement data between different systems, such as the hematology analyzer and the LIS. By storing measurement data in text format and providing scatterplot blood parameters in binary format, it is possible to ensure both readability for human users and efficient processing and analysis by computer models.

FIG. 8 shows, by way of example, the creation of a database from measurement data representing the blood age. Using the instructions in the Cell-Dyn Ruby System Host Interface Specification (LIST NO. 09H05-01 Revision C), the inventors of the patent were able to decrypt the encrypted information in the files and save them as 228 Excel spreadsheets in a folder called “database”. The database consists of two separate folders: a folder containing 149 tables of 2-hour blood samples and another folder containing 79 tables of 24-hour blood samples. This organization facilitates the management and analysis of the data by allowing the separation of samples of different ages.

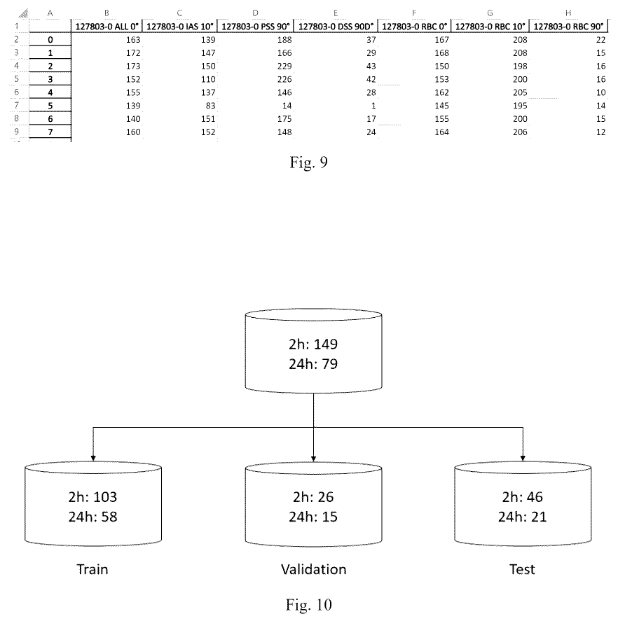

FIG. 9 shows, by example, a section from an Excel table which shows measurement data which have been acquired in the various channels of the hematology analyzer of Abbott Cell-Dyn Ruby. This table was created by transforming the LIS files transferred from the hematology analyzer to the laboratory LIS. This Excel spreadsheet lists the various measurement values for each cell and particle that were collected during the analysis. The organization of the data in such a tabular form allows a simple and clear presentation of the information and facilitates further analysis and processing of the measurement data.

FIG. 10 shows, by example, the division of the measurement data into training, validation and test data sets according to the random principle in the determination of the blood age (<2 h=0, 24 h=1). This random division of the data ensures that the models are not biased to specific patterns within the datasets and that an appropriate representation of the different blood ages within the training, validation and test datasets is ensured. By using separate data sets for training, validation and testing, the models can be continuously evaluated and optimized during the training process and their performance can be assessed on unknown data in the test data set.

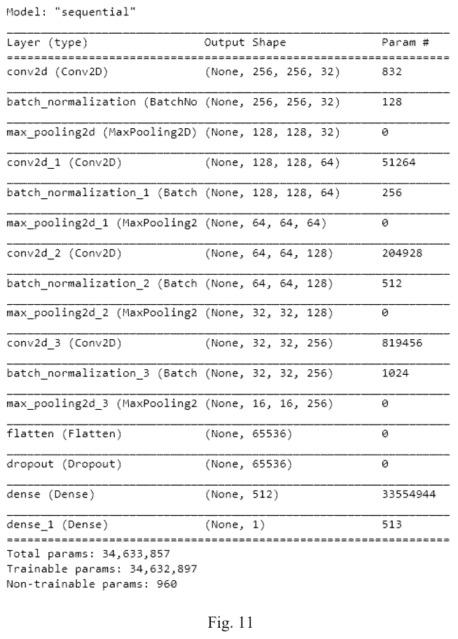

FIG. 11 shows, by example, the architecture of the deep learning model in Keras, which was trained for determining the blood age. In this illustration, one can see the different layers and components of the deep learning model, such as input layers, convolutional layers, activation functions, pooling layers, drop-out layers, and fully interconnected (dense) layers, which are interconnected to build the neural network. The Keras library provides a simple and easy-to-use implementation and customization of the model architecture. By training the model with the prepared training data, the deep learning model can recognize patterns and relationships in the data and thus predict the blood age of unknown samples.

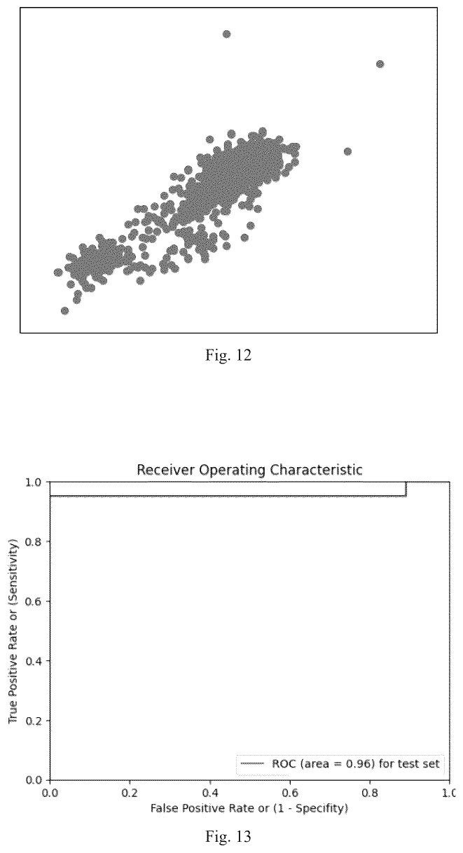

FIG. 12 shows a scatterplot by example.

FIG. 13 shows the area under the Receiver Operating Characteristic (AUROC) curve for the test data set when determining the blood age (<2 h=0, 24 h=1). The AUROC curve is a graphical representation of the performance of a classification model at different classification thresholds. It is often used to assess the diagnostic capability of a model, especially in medical applications. The AUROC curve shows sensitivity (true positive rate) on the vertical axis and specificity (1-false positive rate) on the horizontal axis. A larger area under the curve (AUC) indicates better predictive accuracy of the model. In this example, the AUROC curve shows the ability of the trained deep learning model to correctly determine the blood age (<2 h=0, 24 h=1) on the test data set. A high AUC means that the model is able to distinguish between different blood ages and make precise predictions.

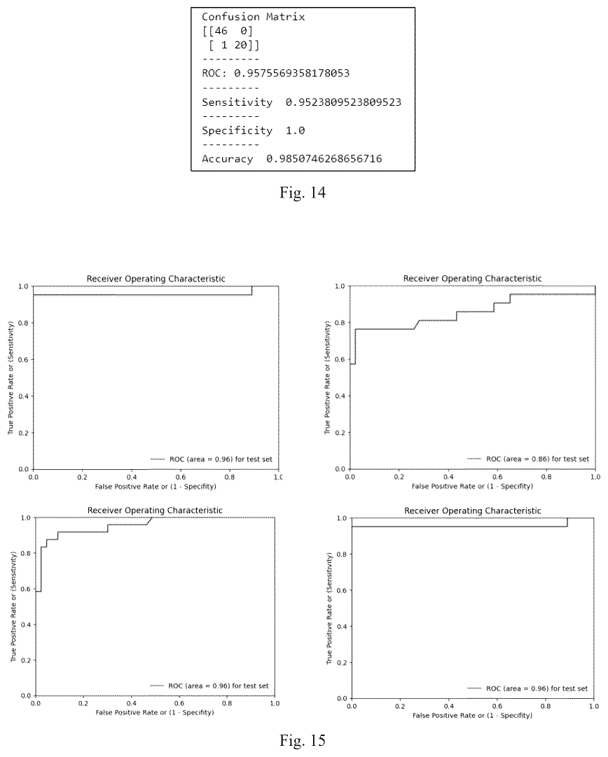

FIG. 14 shows the metrics for the test data set in determining the blood age (<2 h=0, 24 h=1).

FIG. 15 shows the four AUROC curves for the four test folds in the context of the cross-validation method in determining the blood age (<2 h=0, 24 h=1). Cross-Validation is a common method of evaluating the performance of a model by dividing the data into several subsets (in this case four folds). During the cross-validation process, the model is trained on three of the folds and tested on the remaining fold. This process is repeated for all four folds, so that each fold is used exactly once as a test data set. The figure shows the AUROC curves for the four test folds generated during the cross-validation process. Each curve represents the performance of the model in determining the blood age on one of the test folds. The area under the Receiver Operating Characteristic (AUROC) curve shows the diagnostic capability of the model at various classification thresholds. A high AUC indicates that the model has a good ability to differentiate between different blood ages and to make accurate predictions. By analyzing the AUROC curves for the four test folds, the robustness and reliability of the model can be assessed.

1. A computer-implemented method for determining states in vivo, in vitro and/or post-mortem by analyzing blood parameters measured in a hematology analyzer, wherein the method comprises:
obtaining blood parameters of a blood sample by means of a hematology analyzer, wherein the blood parameters comprise quantitative and qualitative measurement variables, wherein the measurement variables comprise the properties of individual cells, wherein the individual cells comprise blood cells, wherein the blood cells comprise white blood cells, red blood cells and platelets, wherein the white blood cells comprise monocytes, lymphocytes, basophils, eosinophils and neutrophils;
creating at least one scatterplot having at least two axes, each axis of the scatterplot comprising a different measurement variable from the obtaining of the blood parameters;
determining at least one in-vivo and/or in-vitro and/or post-mortem state by means of at least one deep learning model, wherein the input variable for the at least one deep learning model comprises at least one scatterplot from the creating of at least one scatterplot; and/or
at least one machine learning model, wherein the input variable for the at least one machine learning model comprises at least one ID vector, wherein the ID vector is created by vectorizing the at least one scatterplot from the creating of at least one scatterplot; and
automatically generating a report which comprises at least one result regarding the determination of the at least one state.

2. A computer-implemented method for automatically generating a report comprising at least one result on the determination of states in vivo, in vitro and/or post-mortem by analyzing blood parameters measured in a hematology analyzer, the method comprising:
obtaining blood parameters of a blood sample by a hematology analyzer, wherein the blood parameters comprise quantitative and qualitative measurement variables, wherein the measurement variables comprise the properties of individual cells, wherein the individual cells comprise blood cells, wherein the blood cells comprise white blood cells, red blood cells and platelets, wherein the white blood cells comprise monocytes, lymphocytes, basophils, eosinophils and neutrophils;
creating at least one scatterplot having at least two axes, each axis of the scatterplot comprising a different measurement variable from the obtaining blood parameters of a blood sample;
determining at least one in vivo and/or in vitro and/or post-mortem state by at least one deep learning model, wherein the input variable for the at least one deep learning model comprises at least one scatterplot from the creating at least one scatterplot; and/or
at least one machine learning model, wherein the input variable for the at least one machine learning model comprises at least one ID vector, wherein the ID vector is created by vectorizing the at least one scatterplot from the creating at least one scatterplot;
automatically generating a report which comprises at least one result regarding the determination of the at least one state; and
transmitting the report from the automatically generating the report using a data signal
receiving the transmitted report.

3. The computer-implemented method according to claim 1, wherein the measurement variables of the individual cells include the number of cells, size, shape, volume, complexity, granularity, electrical conductivity, light scattering at different angles, mean corpuscular volume (MCV), mean corpuscular hemoglobin content (MCH), mean corpuscular hemoglobin concentration (MCHC), red blood cell distribution width (RDW), mean platelet volume (MPV), and/or platelet distribution width.

4. The computer-implemented method according to claim 1, wherein scatterplots are subjected to processing before being analyzed by the at least one deep learning model, the processing comprising size matching, normalization, standardization, noise reduction, test time augmentation (TTA), clustering, contrast matching, and/or filtering individually or in combination.

5. The computer-implemented method according to claim 1, wherein the at least one deep learning model comprises Convolutional Neural Networks, Generative Adversarial Networks, Recurrent Neural Networks, Long Short-Term Memory Networks, Transformer Networks, 3D Convolutional Neural Networks, and/or 4D Convolutional Neural Networks.

6. The computer-implemented method according to claim 1, wherein the at least one 1D vector is subjected to processing prior to analysis by the at least one machine learning model, the processing comprising normalization, standardization, scaling, dimensionality reduction, noise reduction, and feature selection individually or in combination.

7. The computer-implemented method according to claim 6, wherein the dimensionality reduction and/or the feature selection of the at least one ID vector comprises at least one processing method from a group of processing methods comprising the group of processing methods: principal component analysis, T-distributed stochastic neighbor embedding, linear discriminant analysis, truncated singular value decomposition, uniform manifold approximation and projection, independent component analysis, sparse representation, partial least squares regression and kernel principal component analysis.

8. The computer-implemented method according to claim 1, wherein the at least one machine learning model comprises K-Nearest Neighbors, Support Vector Machines, Decision Trees, Random Forests, Multi-Layer Perceptrons, Adaboost Models, Gradient Boosting Models, Naive Bayes, One-Class Support Vector Machines, Isolation Forests, Local Outlier Factors and/or Support Vector Data Descriptions.

9. The computer-implemented method according to claim 1, wherein more than two deep learning models and/or more than two machine learning models and/or a combination of at least one deep learning model and at least one machine learning model comprise an ensemble.

10. The computer-implemented method according to claim 9, wherein the determining of the at least one state is performed using an ensemble technique, the ensemble technique comprising bagging, boosting, stacking, hard voting, soft voting, random subspace, mixture of experts, and/or Bayesian model averaging.

11. The computer-implemented method according to claim 1, wherein in vitro states are based on processes outside a living organism, including changes in the morphology of the blood cells, cell composition, cell function or other features of the individual cells as a result of storage, handling and/or analysis.

12. The computer-implemented method according to claim 1, wherein in vivo states are based on processes within a living organism, including physiological and pathological states such as diseases, biological age, pregnancy, drug action, state of health, nutritional deficiency, hereditary disorders, dehydration, blood clotting disorders, infections and/or anemia.

13. The computer-implemented method according to claim 1, wherein post-mortem states are based on processes of a dead organism, including changes in morphology, cell composition, cell function or other characteristics of the individual cells as a result of diseases, presence of drugs, health status before death, drugs, poisons and/or toxic substances, as well as changes caused by the decay and autolysis of cells and tissues after death.

14. The computer-implemented method according to claim 1, wherein the at least one deep learning model and/or the at least one machine learning model are trained and/or validated on the basis of a prefabricated database, the database comprising measured blood parameters and/or scatterplots, the database being extensible with new measured blood parameters and/or scatterplots for improving performance and accuracy, the database comprising information about known states, diseases or other relevant information contributing to the interpretation and analysis of the measured blood parameters and/or scatterplots.

15. The computer-implemented method according to claim 1, wherein the method is performed to enable integration and use of external data sources, including clinical data, demographic information, medical history and/or genetic data, to provide additional context and improved predictive accuracy in the determination of states.

16. The computer-implemented method according to claim 1, wherein the method comprises supplying real-time blood parameter data from the hematology analyzer to perform continuous monitoring and real-time analysis of states.

17. The computer-implemented method according to claim 1, wherein the method comprises training the at least one deep learning model and/or the at least one machine learning model, wherein the training comprises supervised and/or unsupervised learning, wherein the supervised learning comprises using annotated data in a database to identify patterns and correlations, while the unsupervised learning enables recognition of patterns and correlations in the data of the database without prior annotation to identify novel insights and possibly previously unknown conditions or diseases.

18. The computer-implemented method according to claim 17, wherein the training includes transfer learning, in which pre-trained models from related domains or applications are used as a starting point for training and adaptation to the specific blood parameters and/or scatterplots, in order to increase the efficiency and effectiveness of the training and to reduce the required amount of training data.

19. The computer-implemented method according to claim 17, wherein the training comprises active learning in which the at least one deep learning model and/or the at least one machine learning model selectively search for examples in the database that can most improve their performance and accuracy.

20. The computer-implemented method according to claim 19, wherein the training comprises receiving input from a user for annotation and/or confirmation of the examples to optimize the training process.

21. The computer-implemented method according to claim 17, wherein the training comprises at least one ensemble learning method combining a plurality of learning methods.

22. The computer-implemented method according to claim 17, wherein the training comprises incremental learning in which the deep learning model and/or the machine learning model are continuously and stepwise updated from newly added blood parameters and/or scatterplots in the database.

23. The computer-implemented method according to claim 17, wherein the method performs at least one data augmentation process to increase the size and diversity of the training data in the database to reduce the risk of overfitting.

24. The computer-implemented method according to claim 23, wherein the at least one data augmentation process has a synthetic generation of blood parameters and/or scatterplots which are based on existing data, stochastic methods, statistical models or artificial intelligence algorithms being used in order to generate realistic and representative data for the training of the models.

25. The computer-implemented method according to claim 23, wherein the at least one data augmentation process has at least one transformation of existing blood parameters and/or scatterplots, wherein the at least one transformation has rotations, scales, reflections, shearings, noise and/or distortions, in order to increase the diversity of the training data and to increase the robustness of the determination of the at least one state.

26. The computer-implemented method according to claim 23, wherein the at least one data augmentation process comprises a combination of blood parameters and/or scatterplots from different sources.

27. The computer-implemented method according to claim 23, wherein the at least one deep learning model and/or the at least one machine learning model is adapted to adjust the degree of data augmentation on the basis of boundary conditions, such as the size of the existing database, the number of previous training iterations and/or the current performance and accuracy of the respective model, in order to improve the efficiency of training.

28. A system for determining states in vivo, in vitro and/or post-mortem by analyzing blood parameters measured in a hematology analyzer, comprising a computer device having a computing unit, a memory unit connected thereto and an input unit, wherein the system is designed for:
detecting blood parameters measured in a hematology analyzer, through the input unit, wherein the blood parameters comprise quantitative and qualitative measurement variables, wherein the measurement variables comprise the properties of individual cells, wherein the individual cells comprise white blood cells, red blood cells and platelets, wherein the white blood cells comprise monocytes, lymphocytes, basophils, eosinophils and neutrophils;
producing at least one scatterplot by the computing unit, each axis of the scatterplot comprising a different measurement variable from the detecting of the blood parameters;
determining at least one in vivo and/or in vitro and/or post-mortem state by means of the computing unit by means of at least one deep learning model and/or a machine learning model, wherein the at least one deep learning model receives at least one scatterplot image from step b as input variable, wherein the at least one machine learning model contains at least one ID vector as input variable, wherein the ID vector is generated by vectorizing the at least one scatterplot from the producing of at least one scatterplot;
automatically generating a report by the computing unit, wherein the report comprises at least one result on the determination of the at least one state.

29. The system according to claim 28, wherein the system has a communication interface for transmitting results and reports to other computer systems, laboratory information systems (LIS), hospital information systems (HIS) and/or electronic patient records (EPA).

30. A data signal transmitting the report automatically generated in the method according to claim 1.
